(19) United States
(12) Patent Application Publication
(10) Pub. No.: US 2012/0238908 A1
(43) Pub. Date: Sep. 20, 2012

(54) MASTICATION DETECTION DEVICE AND MASTICATION DETECTION METHOD

(76) Inventors: Keiichi Osako, Tokyo (JP); Toshiyuki Sekiya, Tokyo (JP); Mototsugu Abe, Kanagawa (JP)

(21) Appl. No.: 13/416,493

(22) Filed: Mar. 9, 2012

(30) Foreign Application Priority Data
Mar. 18, 2011 (JP) 2011-061694

(51) Int. Cl. A61B 5/11; A61B 5/12

(52) U.S. Cl. 600/586

(57) ABSTRACT

An information processing apparatus and method provide logic for processing information. In one implementation, an information processing apparatus may include a receiving unit configured to receive an audio signal associated with a motion of a human mandible over a time period. The information processing apparatus may also include a determination unit configured to determine whether the motion of the human mandible corresponds to mastication, based on at least a power of the received audio signal during the time period.

### CROSS-REFERENCE TO RELATED APPLICATIONS

This application is based upon and claims the benefit of priority from Japanese Patent Application JP 2011-061694, filed on Mar. 18, 2011, the entire contents of which are hereby incorporated by reference.

### BACKGROUND

The disclosed exemplary embodiments relate to a mastication detection device and a mastication detection method. In particular, the disclosed exemplary embodiments relate to devices and methods that identify mastication based on a detection of mastication sounds.

The importance of the act of chewing when taking a meal, that is mastication, is being reconsidered due to an increase in health consciousness in recent years. Exemplary advantages obtained by sufficiently masticating are outlined below:

(1) Aids digestion and reduces the burden on the intestines.

(2) Secretion of saliva is promoted and there is prevention of tooth decay.

(3) The jaw is developed and teeth alignment and posture are improved.

(4) It is possible to obtain a sense of fullness due to stimulation of the satiety center, and obesity is suppressed.

Modern food is often soft and there is a tendency for the number of mastications to fall. Accordingly, it is necessary that mastication is performed with awareness in order to obtain a sufficient number of mastications, but realization of this is difficult. Therefore, up to now, a system is proposed where the number of mastications is automatically detected and indicated to the user. For example, a mastication detection device is proposed where a mastication action is detected by attaching a sensor which detects the movement above the temporomandibular joint. However, in a case where a unique sensor such as this is used, there is a problem in that costs increase.

For example, in Japanese Unexamined Patent Application Publication No. 11-123185, a technique is disclosed where a mastication action is detected without a unique sensor by using a cheap and easily obtained microphone. That is, the technique uses an earphone which is also a microphone, detects the sound of a change in shape in the vicinity of an entrance to an ear hole which is generated by mastication by inserting an earphone into an ear, and determines mastication using the detected sound.

### SUMMARY

In Japanese Unexamined Patent Application Publication No. 11-123185, for example, the detection of mastication is performed by comparing the detected sound and a sample sound recorded beforehand. In this case, an error may occur due to the sound sampled beforehand or due to the food which is eaten and the detection of mastication with high accuracy is difficult.

It is desirable to detect mastication with high accuracy at a low cost.

Consistent with an exemplary embodiment, an information processing apparatus includes a receiving unit configured to receive an audio signal associated with a motion of a human mandible over a time period. A determination unit is configured to whether the motion of the human mandible corresponds to mastication, based on at least a power of the received audio signal during the time period.

Consistent with a further exemplary embodiment, a computer-implemented method receives an audio signal associated with a motion of a human mandible over a time period. The method includes determining, using a processor, whether the motion of the human mandible corresponds to mastication, based on at least a power of the received audio signal during the time period.

Consistent with another exemplary embodiment, a tangible, non-transitory computer-readable medium stores instructions that, when executed by at least one processor, cause the processor to perform a method that includes receiving an audio signal associated with a motion of a human mandible over a time period. The method includes determining whether the motion of the human mandible corresponds to mastication, based on at least a power of the received audio signal during the time period.

According to exemplary embodiments of the present disclosure, it is possible to detect mastication with high accuracy at a low cost.

### BRIEF DESCRIPTION OF THE DRAWINGS

FIG. 1 is a block diagram illustrating a configuration example of a mastication detection device according to a first exemplary embodiment of the present disclosure;

FIGS. 2A to 2C are diagrams for describing a process of a mastication sound form calculation section which configures the mastication detection device, in accordance with the first exemplary embodiment;

FIG. 3 is a flowchart illustrating an example of a process sequence of a process for calculating a frame power which is performed by a frame power calculation section of the mastication sound form calculation section, in accordance with the first exemplary embodiment;

FIGS. 4A and 4B are diagrams for describing a process for correcting a power threshold in a power threshold calculation section which configures the mastication detection device, in accordance with the first exemplary embodiment;

FIG. 5 is a flowchart illustrating an example of a process sequence of a process for correcting a power threshold which is performed by the power threshold calculation section, in accordance with the first exemplary embodiment;

FIG. 6 is a diagram for describing a process for determining mastication in a mastication determination section which configures the mastication detection device, in accordance with the first exemplary embodiment;

FIG. 7 is a flowchart illustrating an example of a process sequence of a process for determining mastication which is performed by the mastication determination section, in accordance with the first exemplary embodiment;

FIG. 8 is a block diagram illustrating a configuration example of a system which uses a mastication determination result (detection pulse) of the mastication detection device, in accordance with the first exemplary embodiment;

FIG. 9 is a diagram illustrating an example of a detection pulse which is the mastication determination result output from the mastication determination section of the mastication detection device, in accordance with the first exemplary embodiment;

FIG. 10 is a block diagram illustrating a configuration example of a mastication detection device according to a second exemplary embodiment of the present disclosure;

FIG. 11 is a flowchart illustrating an example of a process sequence of a process for suppressing ambient noise which is performed by an ambient noise suppression section, in accordance with the second exemplary embodiment; and

FIG. 12 is a diagram of an exemplary computer system, consistent with disclosed exemplary embodiments.

### DETAILED DESCRIPTION

Below, exemplary embodiments of the present disclosure will be described. Here, the description will be performed in the order below.

1. First Exemplary Embodiment

2. Second Exemplary Embodiment

3. Modified Example

4. Exemplary Computer Systems

### 1. First Embodiment

### a. Configuration of Mastication Detection Device

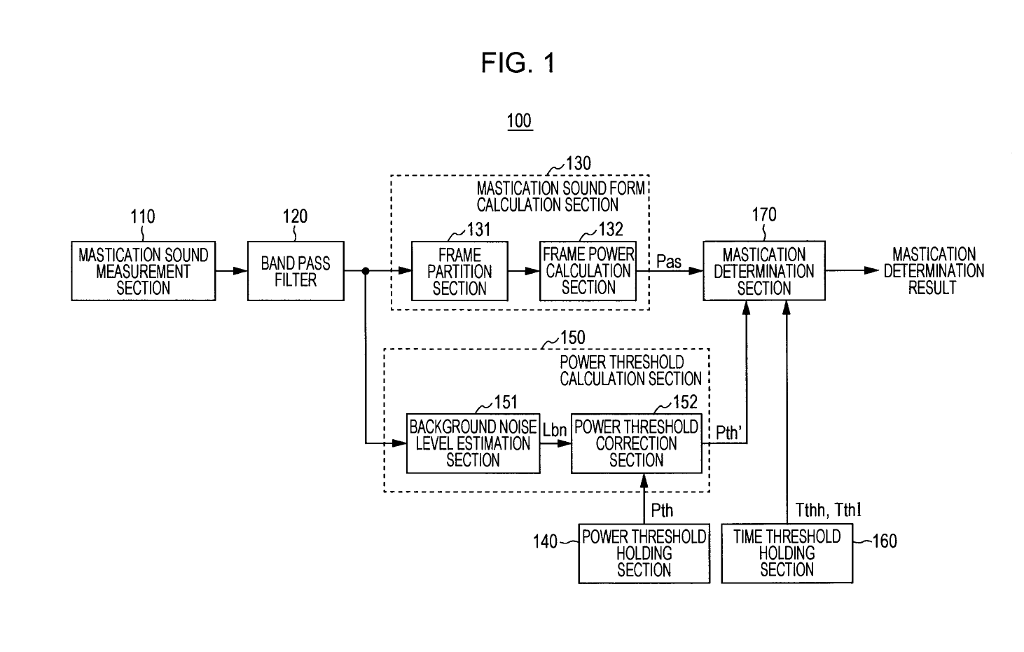

FIG. 1 illustrates a configuration example of a mastication detection device 100 according to a first exemplary embodiment. The mastication detection device 100 has a mastication sound measurement section 110, a band pass filter 120, a mastication sound form calculation section 130, a power threshold holding section 140, a power threshold calculation section 150, a time threshold holding section 160, and a mastication determination section 170.

The mastication sound measurement section 110 measures mastication sound. The mastication sound measurement section 110 is configured by, for example, a microphone, an earphone-type microphone, or the like, and is disposed in an ear of a user or a place which is able to measure mastication sound at a sufficient volume. An output signal of the mastication sound measurement section 110 is, for example, a digital signal with a sampling frequency of approximately 8 kHz.

The band pass filter 120 is a filter for suppressing a component out of the output signal of the mastication sound measurement section 110 which is surplus and is not a mastication sound component and is a filter which only allows a frequency band which includes many mastication sound components to pass. The band pass filter 120 allows, for example, a frequency band of 50 Hz to 200 Hz where there is a sound component generated due to movement of the temporomandibular joint or a frequency band of 900 Hz to 2000 Hz where there is a sound component of teeth hitting against each other, to pass.

The mastication sound form calculation section 130 determines a form Pas of a power transition in the time direction of the output signal of the mastication sound measurement section 110 where surplus signal is suppressed by the band pass filter 120. The mastication sound form calculation section 130 is configured from a frame partition section 131 and a frame power calculation section 132. The frame partition section 131 divides the output signal of the mastication sound measurement section 110 into frames of a predetermined length.

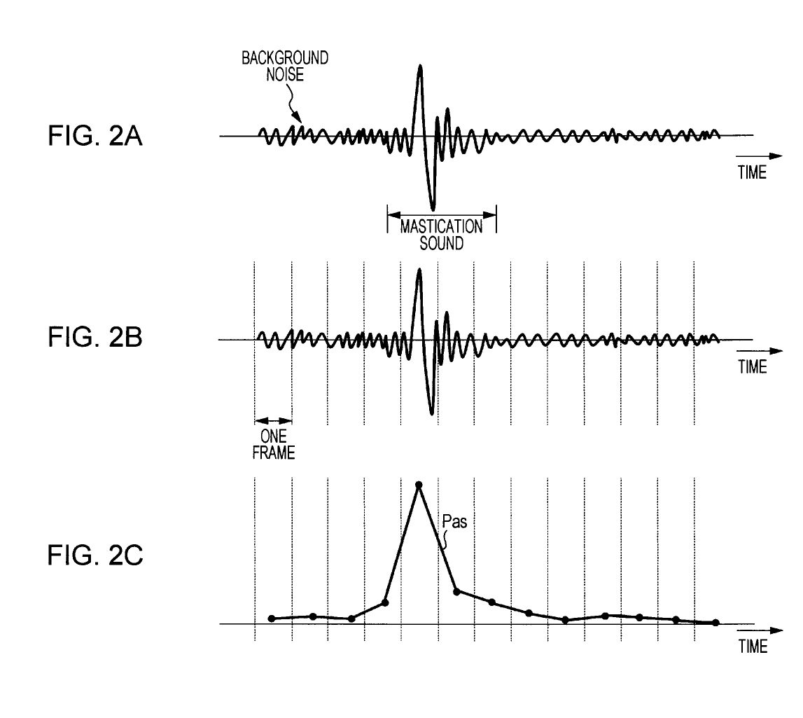

For example, FIG. 2A illustrates an example of the output signal of the mastication sound measurement section 110 over a time period, as a function of time and amplitude. In FIG. 2A, an example is shown where background noise is included. FIG. 2B illustrates an example of a state where the output signal of the mastication sound measurement section 110 over the time period is divided into frames having, for example, an equal frame length. Here, in the example shown in FIG. 2B, an example is shown where there is no overlap between frame sections, but there may be overlapping.

The frame power calculation section 132 determines frame power by calculating an average of the square of each of the sample signals in the time period for each frame. Due to this, the form Pas of the power transition in the time direction of the output signal of the mastication sound measurement section 110 is determined. FIG. 2C illustrates the form Pas of the power transition in the time direction which is determined to correspond to the output signal of the mastication sound measurement section 110 in FIG. 2A.

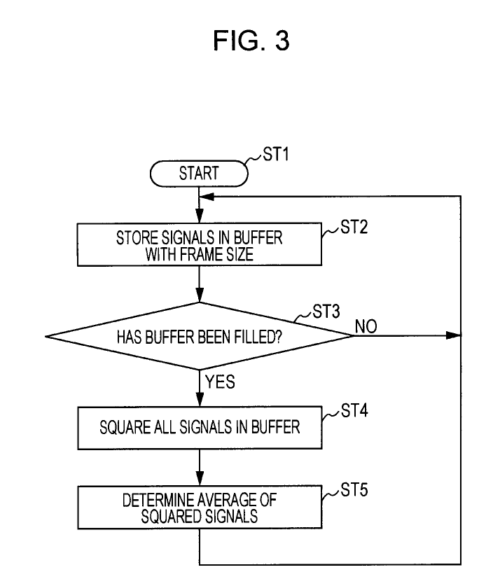

The flowchart of FIG. 3 illustrates an example of a process sequence of a process for calculating the frame power, which is performed by the frame power calculation section 132, in accordance with a disclosed embodiment. The frame power calculation section 132 starts the process in step ST1, and moves to the process of step ST2 after that. In step ST2, the frame power calculation section 132 stores signals of a target frame in a buffer with a frame size.

Next, in step ST3, the frame power calculation section 132 determines whether or not all of the sample signals of the target frame have accumulated in the buffer. When all of the signals have not accumulated, the frame power calculation section 132 returns to the process of step ST2. On the other hand, when all of the signals have accumulated, the frame power calculation section 132 moves to the process of step ST4.

In step ST4, the frame power calculation section 132 squares all of the sample signals in the buffer. Then, in step ST5, the frame power calculation section 132 determines the average of the signals which have been squared and the average is the frame power of the target frame. After the process of step ST5, the frame power calculation section 132 returns to the process of step ST2 and repeats the same process described above with the target frame as the next frame.

Returning to FIG. 1, the power threshold holding section 140 holds a power threshold Pth set in advance which is a parameter which is used by the mastication determination section 170. The power threshold Pth is used in the mastication determination section 170 in order to determine a mastication portion from the form which is determined by the mastication sound form calculation section 130. The power threshold Pth is determined so as to be able to effectively perform determination of the mastication portion by referencing the form which corresponds to the output signal of the mastication sound measurement section 110 of a plurality of people.

The power threshold calculation section 150 estimates a background noise level Lbn and corrects the power threshold Pth which is held in the power threshold holding section 140 based on the background noise level Lbn. The power threshold calculation section 150 is configured from a background noise level estimation section 151 and a power threshold correction section 152. The background noise level estimation section 151 has a buffer which accumulates a sample signal for a previous certain period. The length of the buffer is comparatively longer so as to be able to be stably estimated without being influenced by mastication sound and the like. The background noise level estimation section 151 determines a power average over buffer sections by the same process as the frame power calculation section 132 of the mastication sound form calculation section 130 described above and the power average is set as the background sound level Lbn.

The power threshold correction section 152 corrects the power threshold Pth which is held by the power threshold holding section 140 using the background sound level Lbn estimated by the background noise level estimation section 151 and a corrected power threshold Pth′ is determined. Specifically, the power threshold correction section 152 acquires the corrected power threshold Pth′ by adding the background sound level Lbn to the power threshold Pth.

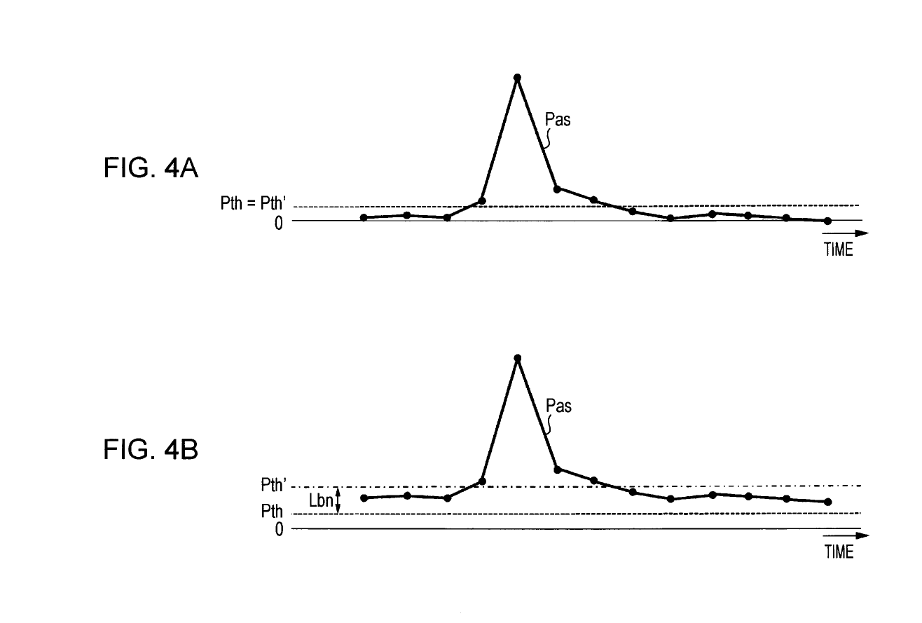

FIG. 4A illustrates an example of a level relationship between the form Pas of the power transition in the time direction of the output signal of the mastication sound measurement section 110 and the power thresholds Pth and Pth′ in a case where there is no background noise, consistent with disclosed embodiments. In this case, the background sound level Lbn is zero and Pth and Pth′ are equal since there is no background noise. FIG. 4B illustrates an example of a level relationship between the form Pas of the power transition in the time direction of the output signal of the mastication sound measurement section 110 and the power thresholds Pth and Pth′ in a case where there is background noise. In this case, the background sound level Lbn is not zero and Pth′=Pth+Lbn since there is background noise. Due to this, the relationship between the power threshold Pth′ and the form Pas is the same as the case where there is no background noise even in the case where there is background noise.

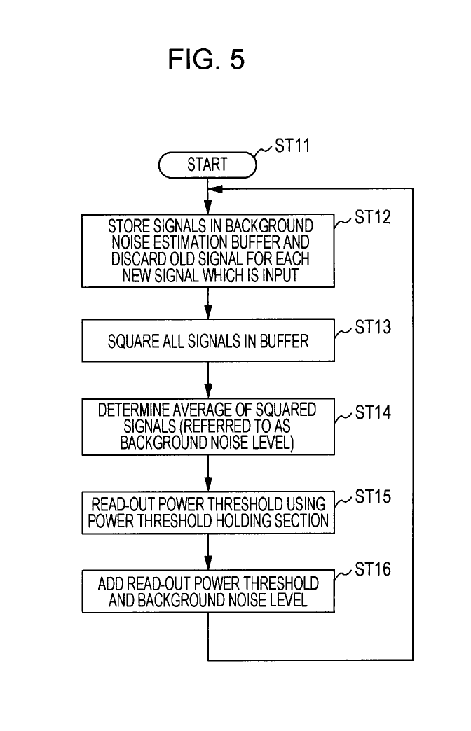

The flowchart of FIG. 5 illustrates an example of a process sequence of a process for correcting the power threshold Pth which is performed by the power threshold calculation section 150, in accordance with a disclosed embodiment. The power threshold calculation section 150 starts the process in step ST11, and moves to the process of step ST12 after that. In step ST12, the power threshold calculation section 150 stores the sample signal in the background noise estimation buffer. In this case, for example, the old sample signals are discarded for each new sample signal which is input.

Next, in step ST13, the power threshold calculation section 150 squares all of the sample signals in the buffer. Then, in step ST14, the power threshold calculation section 150 determines the average of the sample signals which have been squared and the average is the background sound level Lbn. Next, in step ST15, the power threshold calculation section 150 reads out the power threshold Pth from the power threshold holding section 140. Then, in step ST16, the power threshold calculation section 150 acquires the corrected power threshold Pth′ by adding the background sound level Lbn to the power threshold Pth. After the process of step ST16, the power threshold calculation section 150 returns to step ST12 and repeats the same process described above.

Here, in the description above, it has been described that the correction of the power threshold Pth is performed by estimating the background noise level Lbn in the power threshold calculation section 150 even at a time when the mastication sound is measured by the mastication sound measurement section 110. However, the power threshold calculation section 150 may acquire the power threshold Pth′ by performing a correction process described above at a time when the mastication sound is not being measured by the mastication sound measurement section 110. Due to this, it is possible to perform the estimation of the background noise level Lbn without being influenced by the mastication sound and it is possible to increase the accuracy of the process for correcting the power threshold Pth. In this case, the using of the mastication determination result of the mastication determination section 170 in the operational control of the power threshold calculation section 150 is considered.

Returning to FIG. 1, the time threshold holding section 160 holds a time threshold set in advance which is a parameter used by the mastication determination section 170. The time threshold holding section 160 holds an upper limit time threshold Tthh and a lower limit time threshold Tthl where a power larger than the power threshold Pth′ described above is held as a time threshold so as to determine the mastication portion in the form Pas of the power transition in the time direction of the output signal of the mastication sound measurement section 110.

The mastication determination section 170 determines mastication based on the form Pas of the power transition in the time direction of the output signal of the mastication sound measurement section 110 which is determined by the mastication sound form calculation section 130 and outputs the mastication determination result. In this case, the mastication determination section 170 determines the mastication portion based on the form Pas by applying the power threshold Pth′ obtained by the power threshold calculation section 150 and the upper limit time threshold Tthh and the lower limit time threshold Tthl held by the time threshold holding section 160 with regard to the form Pas. Then, the mastication determination section 170 outputs, for example, a detection pulse at a timing when the mastication portion is determined.

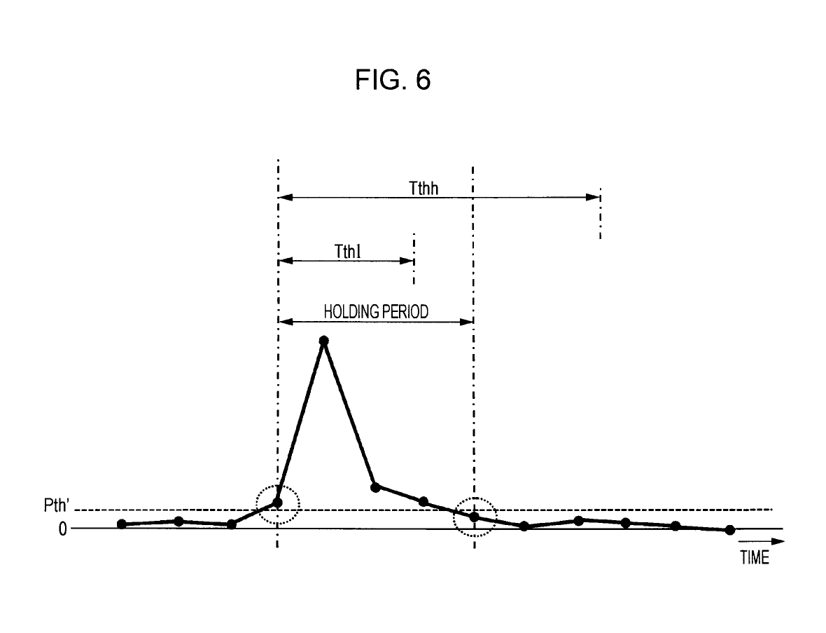

In this case, as shown in FIG. 6, the mastication determination section 170 determines a portion in the form Pas where a power which is larger than the power threshold Pth′ is held for a period between the lower limit time threshold Tthl and the upper limit time threshold Tthh as the mastication portion.

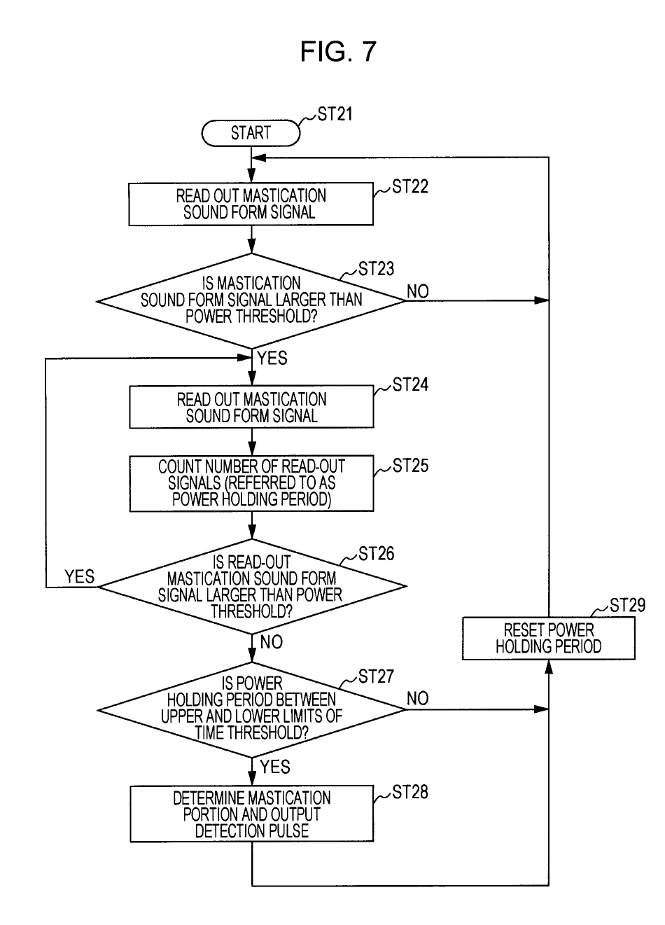

The flowchart of FIG. 7 illustrates an example of a process sequence of a process for determining mastication, which is performed by the mastication determination section 170, in accordance with a disclosed embodiment. The mastication determination section 170 starts the process in step ST21, and moves to the process of step ST22 after that. In step ST22, the mastication determination section 170 reads out a mastication sound form signal obtained from the mastication sound form calculation section 130, that is, a frame power signal which configures the form Pas of the power transition in the time direction of the output signal of the mastication sound measurement section 110.

Next, in step ST23, the mastication determination section 170 determines whether or not the mastication sound form signal (frame power signal) read-out in step ST22 is larger than the power threshold Pth′. When the mastication sound form signal is not larger than the power threshold Pth′, the mastication determination section 170 returns to step ST22, reads out the next mastication sound form signal, and repeats the same process as described above. On the other hand, when the mastication sound form signal is larger than the power threshold Pth′, the mastication determination section 170 moves to the process of step ST24.

In step ST24, the mastication determination section 170 reads out the next mastication sound form signal. Then, in step ST25, the mastication determination section 170 counts the number of read-out mastication sound form signals. That is, the mastication determination section 170 increase the count value by one for each reading out of the mastication sound form signal in step ST24. The count value indicates a power holding period where a state, where the mastication sound form signal (frame power signal) is larger than the power threshold Pth′, is held.

Next, in step ST26, the mastication determination section 170 determines whether or not the mastication sound form signal read-out in step ST24 is larger than the power threshold Pth′. When the mastication sound form signal is larger than the power threshold Pth′, the mastication determination section 170 returns to step ST24, reads out the next mastication sound form signal, and repeats the same process as described above. On the other hand, when the mastication sound form signal is not larger than the power threshold Pth′, the mastication determination section 170 moves to the process of step ST27.

In step ST27, the mastication determination section 170 determines whether the power holding period is accommodated between an upper limit (the upper limit time threshold Tthh) and a lower limit (the lower limit time threshold Tthl) of the time threshold. When the power holding period is not accommodated between the limits, in step ST29, the mastication determination section 170 resets the power holding period, that is, the count value, and after that, returns to the process of step ST22 and repeats the same process described above. On the other hand, when the power holding period is accommodated between the limits, the mastication determination section 170 determines that the power holding period is the mastication portion and outputs the detection pulse in step ST28. After the process of step ST28, in step ST29, the mastication determination section 170 resets the power holding period, that is, the count value, and after that, returns to the process of step ST22 and repeats the same process described above.

The operation of the mastication detection device 100 shown in FIG. 1 will be described. The mastication sound is measured in the mastication sound measurement section 110. The output signal of the mastication sound measurement section 110 is supplied to the mastication sound form calculation section 130 and the power threshold calculation section 150 via the band pass filter 120. In the band pass filter 120, a component out of the output signal of the mastication sound measurement section 110, which is surplus and is not a mastication sound component, is suppressed.

In the mastication sound form calculation section 130, the form Pas of the power transition in the time direction of the output signal of the mastication sound measurement section 110, where the surplus signal has been suppressed by the band pass filter 120, is determined. That is, using the frame partition section 131, the output signal of the mastication sound measurement section 110 is sectioned for each predetermined length of frame length. Then, using the frame power calculation section 132, the frame power is determined by calculating the average of the square of each of the sample signals in the frame for each frame, and due to this, the form Pas of the power transition in the time direction of the output signal of the mastication sound measurement section 110 is determined.

In addition, in the power threshold calculation section 150, the power threshold Pth which is held in the power threshold holding section 140 is corrected based on the background noise level Lbn. That is, in the background noise level estimation section 151, a power average over buffer sections with a predetermined length is determined and the power average is set as the background sound level Lbn. Then, in the power threshold correction section 152, the background sound level Lbn estimated by the background noise level estimation section 151 is added to the power threshold Pth which is held by the power threshold holding section 140 and the corrected power threshold Pth′ is determined.

The form Pas of the power transition in the time direction of the output signal of the mastication sound measurement section 110, which is acquired by the mastication sound form calculation section 130, is supplied to the mastication determination section 170. In addition, the power threshold Pth′ after the correction calculated in the power threshold calculation section 150 and the upper limit time threshold Tthh and the lower limit time threshold Tthl held in the time threshold holding section 160 are supplied to the mastication determination section 170.

In the mastication determination section 170, mastication is determined based on the form Pas of the power transition in the time direction of the output signal of the mastication sound measurement section 110 and the mastication determination result is output. In this case, in the mastication determination section 170, the power threshold Pth′ and the upper limit time threshold Tthh and the lower limit time threshold Tthl are applied with regard to the form Pas. Then, a portion in the form Pas where a power which is larger than the power threshold Pth′ is held for a period between the lower limit time threshold Tthl and the upper limit time threshold Tthh is determined as the mastication portion, and for example, the detection pulse is output.

As described above, in the mastication detection device 100 shown in FIG. 1, the form Pas of the power transition in the time direction of the output signal of the mastication sound measurement section 110 is determined using the mastication sound form calculation section 130. Then, in the mastication determination section 170, mastication is determined by the power threshold Pth′ and the time thresholds Tthh and Tthl being applied to the form Pas. As a result, it is possible to detect mastication with high accuracy at a low cost.

In addition, in the mastication detection device 100 shown in FIG. 1, the background noise level Lbn is estimated by the power threshold calculation section 150. Then, the power threshold Pth which is held in the power threshold holding section 140 is corrected according to the background noise level Lbn and the power threshold Pth′ which is actually used is acquired by the mastication determination section 170. As a result, it is possible to avoid erroneous detection of mastication due to background noise included in the output signal of the mastication sound measurement section 110.

In addition, in the mastication detection device 100 shown in FIG. 1, the band pass filter 120 which sets a frequency band which includes many mastication sound components as a pass band is disposed in the output side of the mastication sound measurement section 110. Then, the surplus component included in the output signal of the mastication sound measurement section 110 is suppressed by the band pass filter 120. As a result, it is possible to avoid erroneous detection of mastication due to a surplus component included in the output signal of the mastication sound measurement section 110.

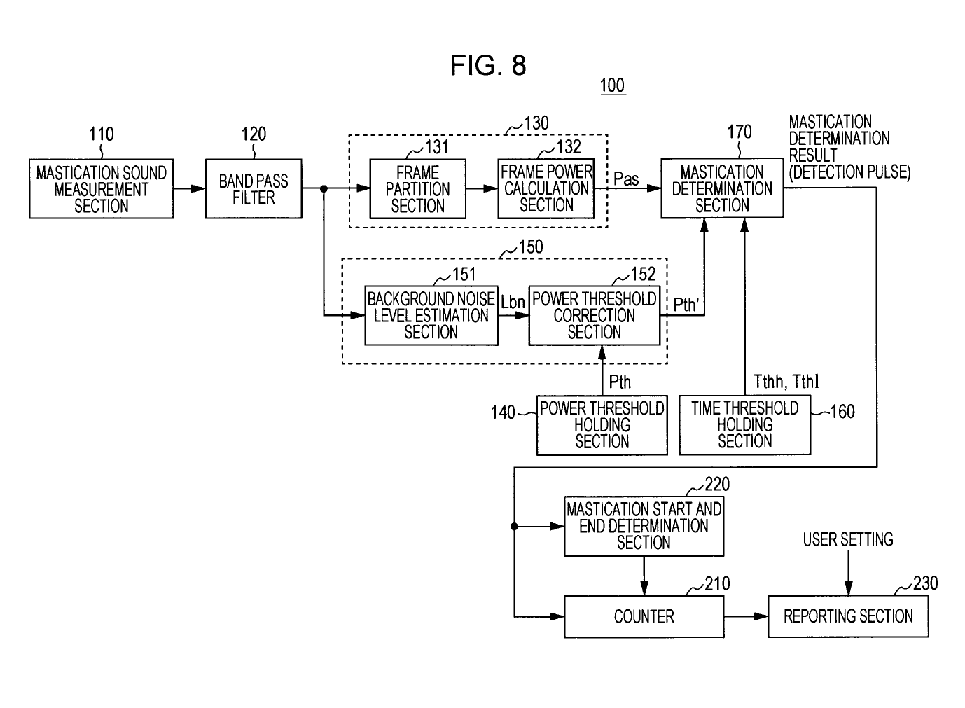

Here, it is possible that the mastication determination result of the mastication detection device 100 shown in FIG. 1 is used in automatic measuring of the number of mastications, a health management system, or the like. FIG. 8 illustrates an example of a configuration of a system, which uses the mastication determination result (detection pulse) of the mastication detection device 100, in accordance with a disclosed embodiment. In FIG. 8, the parts which correspond to FIG. 1 are given the same reference numerals and detail description thereof is omitted.

Other than the mastication detection device 100, the system is provided with a counter 210, a mastication start and end determination section 220, and a reporting section 230. The counter 210 counts the detection pulses which are the mastication determination result output from the mastication detection device 100. The mastication start and end determination section 220 determine the start of mastication and the end of mastication based on the detection pulses which are the mastication determination results output from the mastication detection device 100.

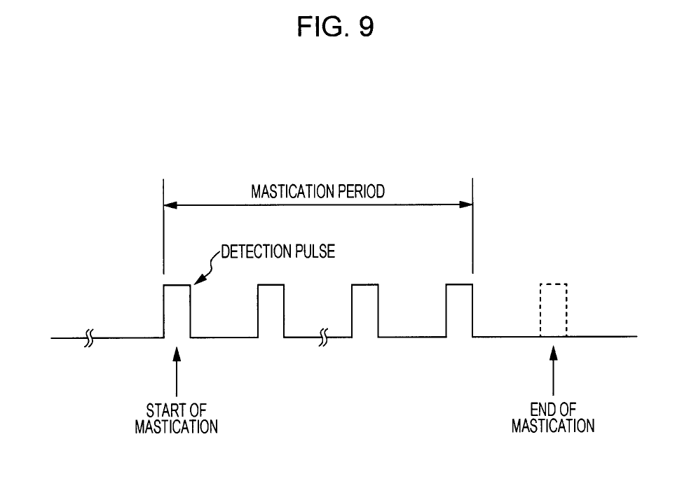

FIG. 9 illustrates an example of a detection pulse which is the mastication determination result output from the mastication determination section 170, in accordance with a disclosed embodiment. From the mastication determination section 170, the output of the detection pulse starts after a mastication period begins, the detection pulse is continuously output after that, and the output of the detection pulse is stopped after the mastication period ends. As a result, the mastication start and end determination section 220 monitors the detection pulse which is output from the mastication determination section 170 and determines that mastication has started when the output of the detection pulse has started. In addition, the mastication start and end determination section 220 monitors the detection pulse which is output from the mastication determination section 170 after the mastication has started and determines that mastication has ended when the output of the detection pulse has stopped.

The counter 210 resets the count value, for example, at a timing when the mastication starts or the mastication ends based on a determination result of the mastication start and end determination section 220. Due to this, the count value of counter 210 in the mastication period shows the number of mastications from the start of mastication. Here, the resetting of the count value of the counter 210 may be configured, for example, so as to be operated by the user at the start of mastication.

The reporting section 230 is provided with a display device such as a liquid crystal panel and a sound producing device such as a speaker or a buzzer, and when the count value of the counter 210 becomes a predetermined value which is set in advance or set by a user, this is reported to the user using the display, sound, or both. Here, the reporting section 230 may be configured so as to report the changes in the count value of the counter 210 at that time to the user using the display, sound, or both.

### 2. Second Embodiment

### a. Configuration of Mastication Detection Device

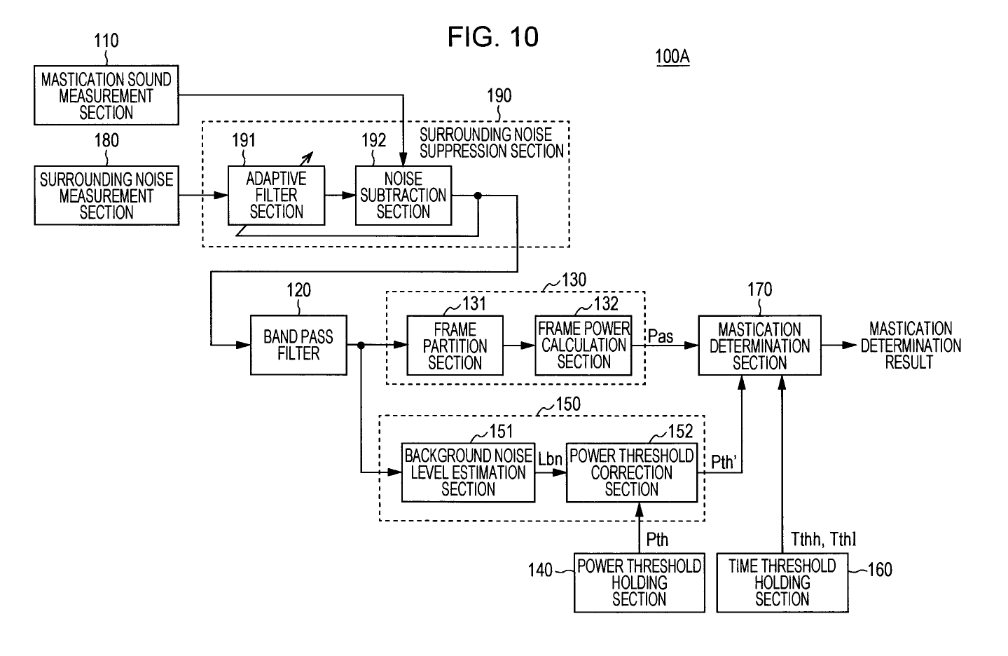

FIG. 10 illustrates a configuration example of a mastication detection device 100A according to a second exemplary embodiment of the present disclosure. The mastication detection device 100A is configured so that an ambient noise measurement section 180 and an ambient noise suppression section 190 are further added to the mastication detection device 100 which is shown in FIG. 1 described above. In FIG. 10, the parts which correspond to FIG. 1 are given the same reference numerals and detail description thereof is omitted. The ambient noise measurement section 180 is disposed in a position which is separate from the mastication sound measurement section 110 and it is necessary to be careful that the mastication sound does not enter the ambient noise measurement section 180.

The ambient noise suppression section 190 suppresses an ambient noise component which is included in the output signal of the mastication sound measurement section 110 with a high degree of accuracy using the output signal of the ambient noise measurement section 180. In the surroundings where the user takes a meal, there are many ambient noises such as from an air conditioner or the like. The suppressing of the ambient noise in this manner is performed because the ambient noise has a negative effect on the mastication detection.

The ambient noise suppression section 190 is configured from an adaptive filter section 191 and a noise subtraction section 192. The adaptive filter section 191 estimates a transfer function from a measurement point in the ambient noise measurement section 180 (ambient noise measurement point) to a measurement point in the mastication sound measurement section 110 (mastication sound measurement point). Here, the mastication sound measurement point has a meaning of a set point in the mastication sound measurement section 110 and the ambient noise measurement point has a meaning of a set point in the ambient noise measurement section 180. The adaptive filter section 191 is typically configured by, for example, a FIR filter or the like. By filtering the output signal of the ambient noise measurement section 180 using the adaptive filter section 191, the ambient noise component at the mastication sound measurement point is estimated with a high degree of accuracy.

In addition, in the adaptive filter section 191, the output signal of the noise subtraction section 192 which will be described later is fed back and a filter coefficient of the adaptive filter section 191 is appropriately changed. A detailed description is omitted but as an adaptive algorithm, there is, for example, a LMS (Least Mean Squares) method, a RLS (Recursive Least Squares) method, and the like.

The noise subtraction section 192 suppresses the ambient noise component included in the output signal of the mastication sound measurement section 110 by performing a subtraction process with the ambient noise component at the mastication sound measurement point which is estimated using the adaptive filter section 191 and the output signal of the ambient noise measurement section 110. The mastication detection device 100A uses a signal after the ambient noise component has been suppressed using the ambient noise suppression section 190 as described above and not by using the output signal of the mastication sound measurement section 110 as it is.

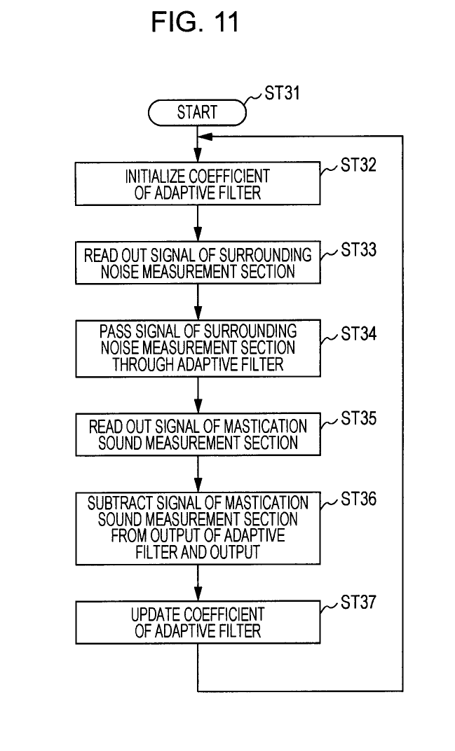

The flowchart of FIG. 11 illustrates an example of a process sequence of a process for suppressing ambient noise which is performed by the ambient noise suppression section 190, in accordance with a disclosed embodiment. The ambient noise suppression section 190 starts the process in step ST31, and moves to the process of step ST32 after that. In step ST32, the ambient noise suppression section 190 initializes the coefficient of the adaptive filter section 191.

Next, in step ST33, the ambient noise suppression section 190 reads out the output signal of the ambient noise measurement section 180. Then, in step ST34, the ambient noise suppression section 190 passes the output signal of the ambient noise measurement section 180 through an adaptive filter.

Next, in step ST35, the ambient noise suppression section 190 reads out the output signal of the mastication sound measurement section 110. Then, in step ST36, the ambient noise suppression section 190 subtracts the output signal of the mastication sound measurement section 110 from the output signal from the adaptive filter and outputs the subtraction result. In step ST37, the ambient noise suppression section 190 changes the coefficient of the adaptive filter by referencing the subtraction output, and after that, returns to step ST33 and repeats the same process as described above.

Other than this, the mastication detection device 100A shown in FIG. 10 is configured in the same manner as the mastication detection device 100 shown in FIG. 1 described above.

The operation of the mastication detection device 100A shown in FIG. 10 will be described. In the mastication sound measurement section 110, the mastication sound is measured. The output signal of the mastication sound measurement section 110 is supplied to the noise subtraction section 192 of the ambient noise suppression section 190. In addition, in the ambient noise measurement section 180, ambient noise such as from an air conditioner is measured. The output of the ambient noise measurement section 180 is supplied to the adaptive filter section 191 of the ambient noise suppression section 190 and the ambient noise component in the mastication sound measuring point is estimated. The ambient noise component estimated in this manner, that is, the output signal of the adaptive filter section 191 is supplied to the noise subtraction section 192.

In the noise subtraction section 192, the subtraction process is performed with the ambient noise component at the mastication sound measurement point which is estimated using the adaptive filter section 191 and the output signal of the ambient noise measurement section 110 and a signal is obtained where the ambient noise component has been suppressed. The output signal of the noise subtraction section 192 is supplied to the mastication sound form calculation section 130 and the power threshold calculation section 150 via the band pass filter 120. A detail description is omitted but hereinafter is the same as the mastication detection device 100 shown in FIG. 1.

As described above, in the mastication detection device 100A shown in FIG. 10, it is possible to obtain the same effects since the configuration is the same as the mastication detection device 100 shown in FIG. 1. In addition, in the mastication detection device 100A shown in FIG. 10, the output signal of the mastication sound measurement section 110 is not used as it is and is used after the ambient noise component has been suppressed with a high degree of accuracy using the ambient noise suppression section 190. As a result, it is possible to avoid erroneous detection of mastication due to the ambient noise component included in the output signal of the mastication sound measurement section.

### 3. Modified Example

Here, in the embodiments described above, the band pass filter 120 is inserted in the output side of the mastication sound measurement section 110, but the band pass filter 120 is not a necessary configuration. In addition, in the embodiments described above, there is a configuration where the power threshold Pth which is held by the power threshold holding section 140 is corrected using the power threshold calculation section 150 and the power threshold Pth′ after correction is used in the mastication detection device 170. However, in surroundings where there is hardly any background noise, this correction is not necessary. In addition, it is possible to consider the omission of the power threshold calculation section 150 by the power threshold Pth which is held by the power threshold holding section 140 being set in advance so as to take the background noise level in consideration.

Here, it is possible for the present disclosure to be configured as per below.

(1) A mastication detection device is provided with a mastication sound measurement section which measures mastication sound, a mastication sound form calculation section which determines a form of power transition in the time direction of an output signal of the mastication sound measurement section, and a mastication determination section which determines mastication based on the form which is determined by the mastication sound form calculation section.

(2) The mastication detection device of (1) where the mastication determination section determines a portion in the form where a power which is larger than a power threshold is held for a period between a lower limit time threshold and an upper limit time threshold as a mastication portion.

(3) The mastication detection device of (2) is further provided with a background noise level estimation section which estimates a background noise level based on the output signal of the mastication sound measurement section, and a power threshold correction section which corrects the power threshold based on the background noise level which is estimated by the background noise level estimation section.

(4) The mastication detection device of (3) where the power threshold correction section obtains the power threshold which is corrected by adding the background noise level which is estimated by the background noise level estimation section to the power threshold which is set in advance.

(5) Any of the mastication detection devices of (1) to (4) is further provided with a band pass filter which is disposed at an output side of the mastication sound measurement section and sets a frequency band which includes many mastication sound components as a pass band, where the mastication sound form calculation section determines the form of the power transition in the time direction of the output signal of the band pass filter.

(6) Any of the mastication detection devices of (1) to (5) is further provided with an ambient noise measurement section which measures ambient noise and an ambient noise suppression section which suppresses an ambient noise component included in the output signal of the mastication sound measurement section based on an output signal of the ambient noise measurement section, where the mastication noise form calculation section determines the form of the power transition of the output signal of the mastication sound measurement section after the ambient noise component is suppressed by the ambient noise suppression section.

(7) The mastication detection device of (6) where the ambient noise suppression section has an adaptive filter which estimates a transfer function from a measurement point in the ambient noise measurement section to a measurement point in the mastication sound measurement section and suppresses the ambient noise component included in the output signal of the mastication sound measurement section by performing a subtraction process with a signal, which is obtained by filtering the output signal of the mastication sound measurement section and the output signal of the ambient noise measurement section using the adaptive filter.

### 4. Exemplary Computer Systems

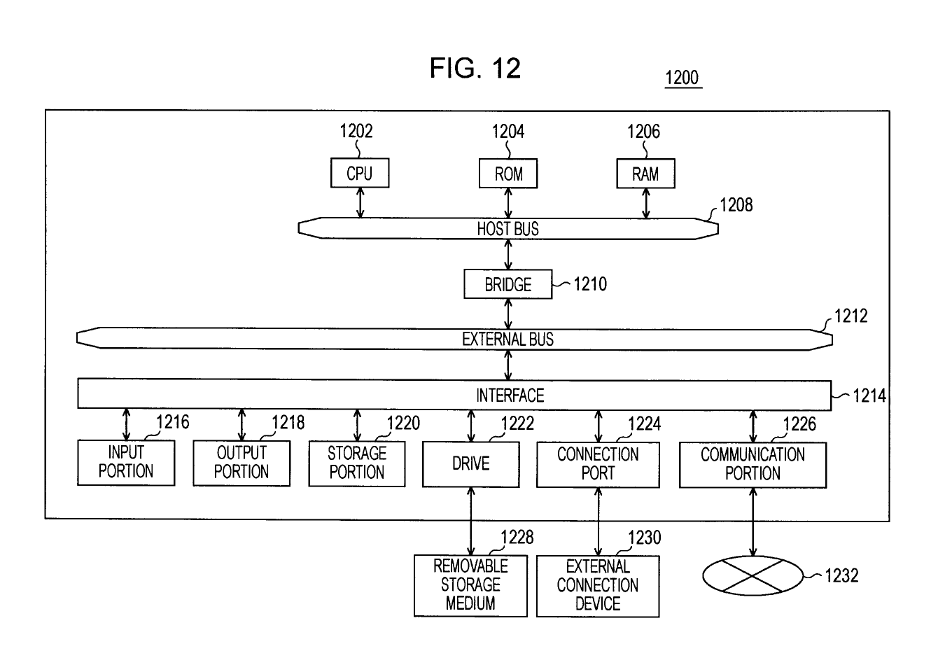

In an embodiment, the functions of the above-described mastication detection device, and the various sections and units, associated with the mastication detection device, can be achieved using, for example, a computer system 1200 shown in FIG. 12. Further, in an additional embodiment, the function of one or more of the structural elements, sections, and units may be achieved by controlling computer system 1200 using instructions stored on a tangible, non-transitory computer-readable storage medium. In such embodiments, examples of computer system 1200 include, but are not limited to a personal computer, a laptop computer, a tablet computer, a mobile phone, a smart phone, a personal digital assistance (PDA), a mobile information terminal, a mobile game console, and/or a head- or ear-mounted specialized computing device.

As shown in FIG. 12, computer system 1200 includes a central processing unit (CPU) 1202, a host bus 1208, a bridge 1210, and a tangible computer-readable storage media, examples of which include a read only memory (ROM) 1204, and a random access memory (RAM) 1206. Furthermore, computer system 1200 includes an external bus 1212, an interface 1214, an input unit 1216, an output unit 1218, a storage unit 1220, a drive 1222, a connection port 1224, and a communication unit 1226.

CPU 1202 may function as an arithmetic processing unit or a control unit, for example, and controls the entire operation or a part of the operation of each structural element based on various instructions stored within ROM 1204, RAM 1206, storage unit 1220, or a removable recording medium 1228. ROM 1204 may be configured to store, for example, a instructions to be loaded on CPU 1202 or data or the like used in an arithmetic operation. RAM 1206 temporarily or permanently stores, for example, instructions to be loaded on CPU 1202 or various parameters or the like arbitrarily changed in execution of a program.

These structural elements are connected to each other by, for example, host bus 1208 capable of performing high-speed data transmission. Host bus 1208 is connected through bridge 1210 to external bus 1212 whose data transmission speed is relatively low, for example. Furthermore, input unit 1216 may include, for example, a mouse, a keyboard, a touch panel, a button, a switch, or a lever. Also, input unit 1216 may be a remote control that can transmit a control signal by using an infrared ray or other radio waves.

Output unit 1218 may be a display device that includes, but is not limited to, a cathode ray tube (CRT), a liquid crystal display (LCD), a plasma display panel (PDP), an electro-luminescence display (ELD), and audio output device (e.g., a speaker or headphones), a printer, a mobile phone, and/or a facsimile, that may provide a visual or auditory notification to a user of acquired information.

Storage unit 1220 is an example of a tangible, non-transitory computer-readable storage medium or device for storing various data. Storage unit 1220 may include, for example, a magnetic storage device such as a hard disk drive (HDD), a semiconductor storage device, an optical storage device, a magneto-optical storage device.

Drive 1222 is a device that reads information recorded on removable recording medium 1228 such as a magnetic disk, an optical disk, a magneto-optical disk, or a semiconductor memory, or writes information in removable recording medium 1228. Removal recording medium 1228 is another example of a tangible, non-transitory storage medium.

Connection port 1224 may be a port that includes, but is not limited to, a USB port, an IEEE13124 port, a SCSI, an RS-232C port, or a port for connecting an externally connected device 1230, such as an optical audio terminal. Externally connected device 1230 may be, for example, a printer, a mobile music player, a digital camera, a digital video camera, or an IC recorder.

Communication unit 1226 is a communication device to be connected to a network 1232, and is, for example, a communication card for a wired or wireless LAN, Bluetooth, or wireless USB, an optical communication router, an ADSL router, or a modem for various types of communication. Network 1232 connected to communication unit 1226 is configured from a wire-connected or wirelessly connected network, and is the Internet, a home-use LAN, infrared communication, visible light communication, broadcasting, or satellite communication, for example.

It should be understood by those skilled in the art that various modifications, combinations, sub-combinations and alterations may occur depending on design requirements and other factors insofar as they are within the scope of the appended claims or the equivalents thereof.

What is claimed is:

1. An information processing apparatus, comprising:
a receiving unit configured to receive an audio signal associated with a motion of a human mandible over a time period; and
a determination unit configured to determine whether the motion of the human mandible corresponds to mastication, based on at least a power of the received audio signal during the time period.

2. The information processing apparatus of claim 1, further comprising a calculation unit configured calculate a representation of the power of the received audio signal during the time period.

3. The information processing apparatus of claim 2, wherein the received audio signal comprises a plurality of discrete audio samples associated with the human mandible motion over the period of time.

4. The information processing apparatus of claim 3, further comprising a frame partition unit configured to:
partition the time period into a plurality of frames, the frames being associated with corresponding temporal boundaries; and
assign subsets of the discrete audio samples to corresponding ones of the frames, based on at least the temporal boundaries.

5. The information processing apparatus of claim 4, wherein at least one of the frames overlaps a portion of an adjacent one of the frames.

6. The information processing apparatus of claim 4, wherein:
the power representation corresponds to a representation of an average power of the received signal as a function of time; and
the calculation unit is further configured to calculate values of the average power of the received signal during corresponding ones of the frames.

7. The information processing apparatus of claim 6, wherein the calculation unit is further configured to:
determine, for at least one of the frames, values indicative of squares of the corresponding discrete audio samples; and
calculate the average power for the at least one frame as a mean of the squared values.

8. The information processing apparatus of claim 6, wherein the determination unit is further configured to obtain at least one of a threshold power value, a first threshold time period, or a second threshold time period.

9. The information processing apparatus of claim 8, wherein the determination unit is further configured to:
determine, for at least one of the frames, that a corresponding value of the average power exceeds the threshold power value; and
identify a time period during which the average power exceeds the threshold time period.

10. The information processing apparatus of claim 9, wherein the determination unit is further configured to:
determine whether the identified time period exceeds the first threshold time period, and fails to exceed the second threshold time period; and
determine that the motion of the human mandible corresponds to mastication, when the identified time period exceeds the first threshold time value, and fails to exceed the second threshold time period.

11. The information processing apparatus of claim 8, further comprising a threshold power calculation unit configured to generate the threshold power value.

12. The information processing unit of claim 11, where the threshold power calculation unit is further configured to:
identify an initial value of the threshold power value;
compute an average power of background noise during at least the time period associated with the received audio signal; and
adjust the initial value in accordance with the average power of the background to generate the threshold average power.

13. The information processing unit of claim 12, wherein the threshold power calculation unit is further configured to:
obtain an audio signal corresponding to background noise, the audio signal being measured absent mastication; and
compute the average power of background noise, based on the audio signal corresponding to the background noise.

14. The information processing unit of claim 8, further comprising a time threshold determination unit configured to determine at least one of the first threshold time value or the second threshold time value.

15. The information processing unit of claim 14, wherein the time threshold determination unit is further configured to obtain at least one of the first threshold time value or the second threshold time value.

16. The information processing unit of claim 1, further comprising a detection unit configured to:
detect sounds associated with the motion of the human mandible; and
generate the audio signal based on the detected sounds.

17. The information processing unit of claim 1, further comprising a reporting unit configured to generate a signal to display an indication to a user that the human mandible motion corresponds to mastication.

18. The information processing apparatus of claim 1, wherein the average power representation comprises a graphical representation of the average power of the received signal.

19. A computer-implemented method, comprising:
receiving an audio signal associated with a motion of a human mandible over a time period; and
determining, using a processor, whether the motion of the human mandible corresponds to mastication, based on at least a power of the received audio signal during the time period.

20. A tangible, non-transitory computer-readable medium storing instructions, that when executed by at least one processor, causes the at least one processor to perform a method, comprising:
receiving an audio signal associated with a motion of a human mandible over a time period; and
determining whether the motion of the human mandible corresponds to mastication, based on at least a power of the received audio signal during the time period.
